(19) United States
(12) Patent Application Publication
(10) Pub. No.: US 2026/0306535 A1
(43) Pub. Date: Oct. 8, 2026

(54) Methods for Increasing Healing Speed of Human and Animal Wounds

(71) Applicant: Courtland IMEL, Farmers Branch, TX (US)

(72) Inventor: Courtland IMEL, Farmers Branch, TX (US)

(21) Appl. No.: 19/715,339

(22) Filed: Jun. 22, 2026

Related U.S. Application Data
(63) Continuation of application No. 18/124,657, filed on Mar. 22, 2023.
(60) Provisional application No. 63/322,797, filed on Mar. 23, 2022.

(51) Int. Cl. A61K 33/30; A61K 33/06; A61P 17/02

(52) U.S. Cl. CPC A61K 33/30; A61K 33/06; A61P 17/02

(57) ABSTRACT

A novel composition of ions, and the use of the composition of ions in speeding up the healing process of mammal wounds, including human wounds, with a reduction of scarring. In one exemplary embodiment the composition of ions includes a formula of Rubidium, Calcium, Zinc, and Potassium. Rubidium is absolutely necessary to ensure the speed of the healing process. These specific combination of ions are referred to as a Protease Down Regulator (PDR).

### RELATED APPLICATION

This application is a continuation-in-part of U.S. patent application U.S. Ser. No. 18/124,657 filed Mar. 22, 2023, entitled Novel Methods for Increasing Healing Speed of Human and Animal Wounds, which claims convention priority of U.S. Provisional Patent Application U.S. Ser. No. 63/322,797 filed Mar. 23, 2022, entitled Novel Methods for Increasing Healing Speed of Human Wounds, the teachings of which are incorporated herein by reference.

### TECHNICAL FIELD

This disclosure is directed to a composition for treatment of mammal wounds including in humans and animals.

### BRIEF DESCRIPTION OF FIGURES

The drawing figures depict one or more implementations, by way of example only, not by way of limitations. In the figures, like reference numerals refer to the same or similar elements.

FIG. 1 is a picture of an application of a bandage with a wound cream to an open wound of a human; and

FIG. 2 illustrates a method of treating a wound of a human with the wound cream.

### DETAILED DESCRIPTION

Reference now is made in detail to the examples illustrated in the accompanying drawings and discussed below.

The disclosure describes a more rapid wound healing composition of select ions, and the use of the composition of ions in speeding up the healing process of mammal wounds with a reduction of scarring. Several inventive embodiments of the present disclosure are described below. In one exemplary embodiment the composition of ions includes Rubidium, Calcium, Zinc, and Potassium. The specific composition of ions is a formula and is referred to as a Protease Down Regulator (PDR). Rubidium is necessary to help speed up the wound healing process.

Additional objects, advantages and novel features of the examples will be set forth in part in the description which follows, and in part will become apparent to those skilled in the art upon examination of the following and the accompanying drawings or may be learned by production or operation of the examples. The objects and advantages of the present subject matter may be realized and attained by means of the methodologies, instrumentalities and combinations particularly pointed out in the appended claims.

In the following detailed description, numerous specific details are set forth by way of examples in order to provide a thorough understanding of the relevant teachings. However, it should be apparent to those skilled in the art that the present teachings may be practiced without such details. In other instances, well-known methods, procedures, components, and circuitry have been described at a relatively high-level, without detail, in order to avoid unnecessarily obscuring aspects of the present teachings.

The composition of ions in the PDR, referred to herein as PDR ions, are a wound management factor that normalizes aberrant biochemistry with non-healing wounds. Testing of PDR in cellular assays indicates that PDR acts on the gene expression level by down-regulating the genes for matrix metal proteinases and pro inflammatory cytokines, and up-regulates the genes for growth factors and tissue inhibitors of matrix metal proteinases. These four gene regulatory effects result in advantageous biochemical changes in the wound of human tissue toward a healing trajectory.

The present disclosure includes the novel composition of ions and a method of use in a wound cream that regulates the healing cascade at the gene expression level. The wound cream may include a petrolatum formulation or other suitable formulation including the composition of ions. Suitable formulations may include saline solution, water soluble based creams, oil base creams, and applications within a bandage. The PDR ions regulate the ability to heal the wound faster by increasing epithelialization and granulation, while reducing edema, inflammation, and exudate accumulation. Through human studies on 11 patients, the use of the PDR ions in the wound cream showed complete wound closure within 4 days, as compared to a hydrogel that closed the wound in 10 days. This process shows a 60% faster wound closure rate. The method involves the PDR ions that is a mixture of Rubidium, Calcium, Zinc, and Potassium in the wound cream. In a non-limiting exemplary embodiment, the composition of ions in the wound cream can be between 5.000000 to 10% Rubidium, 2.000000 to 10% Calcium, 3.00000 to 10% Zinc, and 4.000000 to 10% Potassium. The remaining portion of the wound cream is the petrolatum formulation to make up 100%. These percentages are critical as a formula as they have a synergy effect and contribute to the exceptional healing time of the wound within 4 days. It is noted that, for example, the listed percentages can be represented in parts per million (ppm) by multiplying the listed percentage by 10,000. Thus, 5.000000% is 50000 ppm and so forth.

This formulation is not found in nature. Sea water, for instance, provides some healing capabilities, contains the following ions and concentrations according to the American Chemical Society and a Stanford Sea Urchin Study:

| | | | Mineral Makeup | | Product | |
|---|---|---|---|---|---|---|
| | American | | of Sea Water, | | Minimum | |
| | Chemical | | Stanford Urchin | | Concentrations | |
| Source | Society | | Study, p. 1 | | in ppm | |
| Metal Ion | Ionic | | Ionic Concentration | | Ionic Concentration | |
| | Concentration | | | | | |
| Sodium (Na⁺) | 10560 | ppm | 10561 | ppm | 0.0 | ppm |
| Magnesium | 1260 | ppm | 1272 | ppm | 0.0 | ppm |
| (Mg²⁺) | | | | | | |
| Calcium (Ca²⁺) | 400 | ppm | 400 | ppm | 20000 | ppm |
| Potassium (K⁺) | 380 | ppm | 380 | ppm | 40000 | ppm |
| Strontium (Sr²⁺) | 13 | ppm | 13 | ppm | 0.0 | ppm |

| | | | | | |
|---|---|---|---|---|---|
| Sulfur | Not provided | 884 | ppm | 0.0 | ppm |
| Rubidium | Not provided | 0.2 | ppm | 50000 | ppm |

| | | | | |
|---|---|---|---|---|
| Zinc | Not provided | not provided | 30000 | ppm |

It is known that sea water does provide healing, but it is not known for rapid healing of a wound. Further, sea water does contain bacteria, that may increase the infection within the wound and delay in healing.

### Study

A study was performed on 11 human male patients with a mean age of 20.5. The study was a randomized, double blind, placebo controlled clinical trial. All subjects were healthy Caucasian males without a history of illness, infection, or pre-existing condition that may affect wound healing. The study involved 9 non-smokers and 2 smokers who reported smoking less than one pack per day.

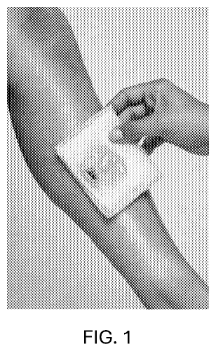

Four similar wounds were placed on each subject: two wounds having a depth of 1 centimeter (cm) were created using a sterile scalpel on each the right thigh and the left thigh. The wounds were placed, one proximal and one distal on each of the ventral aspects of the thighs. A computer program generated the type of treatment to be used on each wound site for each individual. FIG. 1 illustrates a bandage formed of PDR material with the composition of ions in the wound cream that is placed on the bandage and applied to the created human wounds. In this example, a petrolatum formulation containing 5.00000% Rubidium 2.000000% Calcium, 3.00000% Zinc, and 4.00000% Potassium is applied to the wounds.

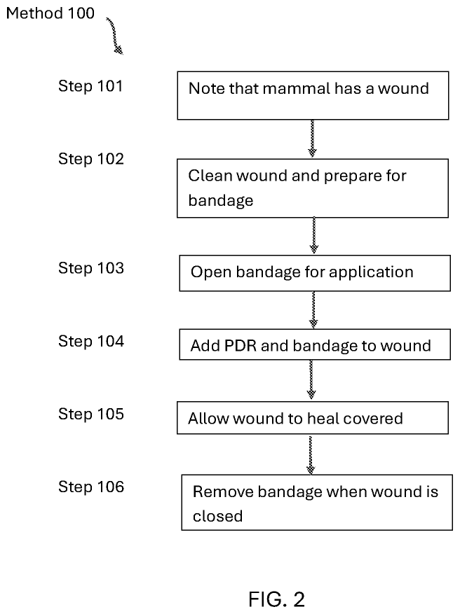

FIG. 2 illustrates method 100 having steps for treating each wound of a mammal.

At step 102, the wound is cleaned with sterile material.

At step 104, the composition of ions in the wound cream is applied to the wounds. In this example, a petrolatum formulation containing 5.00000% Rubidium, 2.00000% Calcium, 3.00000% Zinc, and 4.00000% Potassium is applied to the wounds. The wound is covered with the PDR material and then covered with the bandage to allow healing to occur. The PDR can be embedded in the bandage.

### Summary of Wound Re-Epithelialization

TABLE 1

| | Day After Wounding | | | | | | | | | |
|---|---|---|---|---|---|---|---|---|---|---|
| Treatment | 1 | 2 | 3 | 4 | 5 | 6 | 7 | 8 | 9 | 10 |
| Telfa | 0.00 | 0.00 | 0.00 | 0.00 | 0.36 | 0.09 | 0.45 | 0.73 | 0.82 | 0.73 |
| PEG Based | 0.00 | 0.00 | 0.18 | 0.09 | 0.64 | 0.27 | 0.36 | 0.64 | 0.55 | 1.00 |
| Placebo | | | | | | | | | | |
| Bencelok | 0.00 | 0.00 | 0.36 | 0.18 | 0.73 | 0.64 | 0.91 | 0.82 | 1.30 | 1.30 |
| Hydrogel | | | | | | | | | | |
| PDR Cream | 0.00 | 0.00 | 1.20 | 1.50 | 2.50 | 3.40 | 3.50 | 3.50 | 3.60 | 3.60 |

Average Measured Epithelialization on 11 patients. A value equal to 1.5 indicates wound closure and a value greater than 4 indicates complete wound healing. As shown in Table 1, the wound is closed within 4 days and completely healed by day 6.

### Summary of Granulation Tissue Formation

TABLE 2

| | Day After Treatment | | | | | | | |
|---|---|---|---|---|---|---|---|---|
| Treatment | 3 | 4 | 5 | 6 | 7 | 8 | 9 | 10 |
| Telfa | 0.18 | 0.09 | 0.00 | 0.00 | 0.27 | 0.36 | 0.73 | 0.64 |
| PEG Based | 0.18 | 0.00 | 0.09 | 0.00 | 0.09 | 0.46 | 0.55 | 0.91 |
| Placebo | | | | | | | | |
| Bencelok | 0.36 | 0.09 | 0.18 | 0.46 | 0.55 | 0.55 | 0.91 | 0.91 |
| Hydrogel | | | | | | | | |
| PDR Cream | 0.82 | 1.00 | 1.00 | 1.00 | 1.00 | 1.30 | 0.82 | 1.00 |

Average Measured granulation tissue formation on 11 subjects. A value of 1.0 shows a closed wound. A value greater than 1 shows a mixture of scab with covering. As shown in Table 2, the use of the PDR wound cream showed complete wound closure within 4 days, as compared to a hydrogel that closed the wound in 10 days. This process shows a 60% faster wound closure rate. As the concentration of these ions in the formula is increased from these critical percentages, we found a slight increase in the closure rate.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

1. A composition of ions configured to increase a healing speed of a wound of a mammal, wherein the composition of ions comprises a protease down regulator (PDR).

2. The composition of claim 1, wherein the composition of ions comprises Rubidium, Calcium, Zinc, and Potassium.

3. The composition of claim 2, wherein the composition of ions comprises a mixture of 50,000 parts per million (ppm) to 100,000 ppm Rubidium, 20,000 ppm to 100,000 ppm Calcium, 30,000 ppm to 100,000 ppm Zinc, and 40,000 ppm to 100,000 ppm Potassium.

4. The composition of claim 2, wherein the PDR composition of ions is configured to regulate an ability to heal the wound faster by increasing epithelialization and granulation.

5. The composition of claim 2, wherein the composition of ions is configured to reduce edema, inflammation, and exudate accumulation of the mammal.

6. The composition of claim 2, wherein the composition of ions is configured to close the wound within 4 days.

7. The composition of claim 2, wherein the composition of ions is suitable for topical administration on skin of a human.

8. A method of treating a wound of a human, comprising:
applying a composition of ions to the wound, wherein the composition of ions comprises a protease down regulator (PDR).

9. The method of claim 8, wherein the composition of ions comprises Rubidium, Calcium, Zinc, and Potassium.

10. The method of claim 9, wherein the composition of ions comprises a mixture of 50,000 parts per million (ppm) to 100,000 ppm Rubidium, 20,000 ppm to 100,000 ppm Calcium, 30,000 ppm to 100,000 ppm Zinc, and 40,000 ppm to 100,000 ppm Potassium.

11. The method of claim 9, wherein the PDR composition of ions regulates an ability to heal the wound faster by increasing epithelialization and granulation.

12. The method of claim 9, wherein the composition of ions reduces edema, inflammation, and exudate accumulation.

13. The method of claim 9, wherein the composition of ions closes the wound within 4 days.

14. The method of claim 9, wherein the composition of ions is suitable for topical administration on skin of the human.
